(19) United States
(12) Patent Application Publication
(10) Pub. No.: US 2026/0207237 A1
(43) Pub. Date: Jul. 23, 2026

(54) IMPLANTS, INSTRUMENTS, AND METHODS FOR MINIMALLY INVASIVE BUNION PROCEDURES

(71) Applicant: Paragon 28, Inc., Englewood, CO (US)

(72) Inventors: Albert DACOSTA, Lone Tree, CO (US); Sean Patrick GILL, Denver, CO (US); Kaitlin KARAS, Denver, CO (US); Lucas Charles MCMAHAN, Lone Tree, CO (US); Kenneth Allan ROGGOW, Denver, CO (US); Tristan COLLETTE, Denver, CO (US); Spanky RAYMOND, Uniontown, OH (US)

(73) Assignee: Paragon 28, Inc., Englewood, CO (US)

(21) Appl. No.: 19/572,512

(22) Filed: Mar. 19, 2026

Related U.S. Application Data
(63) Continuation of application No. PCT/US2024/047485, filed on Sep. 19, 2024.
(60) Provisional application No. 63/584,073, filed on Sep. 20, 2023.
(60) Provisional application No. 63/618,457, filed on Jan. 8, 2024.

(51) Int. Cl. A61B 17/88; A61B 17/17; A61B 17/56

(52) U.S. Cl. CPC A61B 17/8866; A61B 17/1775; A61B 2017/565

(57) ABSTRACT

An instrument having a proximal portion, that includes a guide having at least one opening, the at least one opening having at least one trajectory. The instrument includes a distal portion that has an engagement mechanism that engages with a first segment of a first metatarsal, and an adjustment mechanism that engages with a second segment of a first metatarsal. A surgical method is disclosed with the steps of: coupling the instrument to the foot of a patient; positioning the instrument so a pair of rails are substantially parallel to the first metatarsal; manipulating a locking mechanism; coupling the distal portion of the instrument with a proximal portion segment of the first metatarsal; coupling an angel wing with the instrument; correcting the deformity of the first metatarsal; targeting the implantation of at least one implant and implanting the at least one implant into the first metatarsal.

### CROSS RELATED APPLICATIONS

This is a bypass continuation application which claims priority of International PCT Application No. PCT/US 2024/047485 filed Sep. 19, 2024, and entitled “Implants, Instruments, and Methods For Minimally Invasive Bunion Procedures,” which claims benefit of priority to U.S. Provisional Application No. 63/584,073 filed on Sep. 20, 2023, and entitled “Implants, Instruments, and Methods For Minimally Invasive Bunion Procedures,” and U.S. Provisional Application No. 63/618,457 filed on Jan. 8, 2024, and entitled “Implants, Instruments, and Methods For Minimally Invasive Bunion Procedures,” the disclosures of all of these applications are hereby incorporated herein by reference in their entirety.

### TECHNICAL FIELD

The present disclosure relates to surgical implants, instruments, and methods of use to be implemented in surgical procedures. The present disclosure relates to podiatric and orthopedic surgical implants, instruments, and methodology to be implemented in various procedures of the foot and/or ankle, for example procedures to address deformities of the forefoot and/or midfoot. More specifically, but not exclusively, the present disclosure relates to surgical implants, instruments, and methodology to be implemented procedures to correct deformities of the first metatarsal.

### BACKGROUND OF THE INVENTION

Many currently available surgical implants, instruments and guides, as well as methodology, do not completely address the needs of patients. Additionally, many currently available surgical implants, instruments, guides, and methodology fail to account for properties of joint anatomy and accordingly can decrease favorability of the outcome for the patient.

### SUMMARY OF THE INVENTION

The present disclosure is directed toward implants, instrumentation, guides, and methodology to be implemented in procedures of the foot and ankle, including the ankle joint.

A first aspect of the present disclosure is an instrument. The instrument includes a proximal portion, which includes a guide having at least one opening, the at least one opening having at least one trajectory. The instrument also includes a distal portion. The distal portion includes an engagement mechanism configured to engage with a first segment of a first metatarsal of a patient, and an adjustment mechanism configured to engage with a second segment of a first metatarsal of the patient.

A second aspect of the present disclosure is an instrument having a proximal portion that includes a guide translatable about an elongated opening and pivotable about a longitudinal axis. The guide has at least one opening, with the at least one opening having at least one trajectory. The guide also has a distal portion that includes an engagement mechanism configured to engage with a first segment of the first metatarsal of a patient. The engagement mechanism has a first segment and a second segment separated from the first segment by a substantially 90-degree angle with the second segment including a texture along at least a portion of a defined length. The engagement mechanism also includes an adjustment mechanism configured to engage with a second segment of the first metatarsal of the patient. The adjustment mechanism has a first opening, a plurality of slots, a second opening and a locking gate.

A third aspect of the present disclosure is a surgical method having the steps of coupling an instrument to the foot of a patient, positioning the instrument such that a pair of rails are substantially parallel to a long axis of a first metatarsal, The method further includes manipulating a locking mechanism of the instrument from a closed position to an open position and coupling a distal portion of the instrument with a proximal portion segment of the first metatarsal. The method further includes the steps of correcting a deformity of the first metatarsal and targeting for the implantation of at least one implant and implanting at least one implant into the first metatarsal.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a top, front perspective view of an exemplary orthopedic system shown adjacent to the anatomy of the foot, in accordance with the present disclosure;

FIG. 2 is a top, rear perspective view of the exemplary orthopedic system of FIG. 1 shown adjacent to the anatomy of the foot, in accordance with the present disclosure;

FIG. 3 is a top view of the exemplary orthopedic system of FIG. 1 shown adjacent to the anatomy of the foot, in accordance with the present disclosure;

FIG. 4 is a front view of the exemplary orthopedic system of FIG. 1 shown adjacent to the anatomy of the foot, in accordance with the present disclosure;

FIG. 5 is a side view of the exemplary orthopedic system of FIG. 1 shown adjacent to the anatomy of the foot, in accordance with the present disclosure;

FIG. 6 is a rear view of the exemplary orthopedic system of FIG. 1 shown adjacent to the anatomy of the foot, in accordance with the present disclosure;

FIG. 7 is an alternate side view of the exemplary orthopedic system of FIG. 1 shown adjacent to the anatomy of the foot, in accordance with the present disclosure;

FIG. 8 is a top, front perspective view of the exemplary orthopedic system of FIG. 1, in accordance with the present disclosure;

FIG. 9 is a top, rear perspective view of the exemplary orthopedic system of FIG. 1, in accordance with the present disclosure;

FIG. 10 is a top view of the exemplary orthopedic system of FIG. 1, in accordance with the present disclosure;

FIG. 11 is a rear view of the exemplary orthopedic system of FIG. 1, in accordance with the present disclosure;

FIG. 12 is a side view of the exemplary orthopedic system of FIG. 1, in accordance with the present disclosure;

FIG. 13 is a front view of the exemplary orthopedic system of FIG. 1, in accordance with the present disclosure;

FIG. 14 is an alternate side view of the exemplary orthopedic system of FIG. 1 shown adjacent to the anatomy of the foot, in accordance with the present disclosure;

FIG. 15 is an exploded view of the exemplary orthopedic system of FIG. 1, in accordance with the present disclosure;

FIG. 16 is a top view of an exemplary implant system adjacent to the anatomy of the foot, in accordance with the present disclosure;

FIG. 17 is a side view of the exemplary implant system of FIG. 16 shown adjacent to the anatomy of the foot, in accordance with the present disclosure;

FIG. 18 is an elevated side perspective view of the exemplary implant system of FIG. 16, in accordance with the present disclosure;

FIG. 19 is a rear view of an exemplary implant of the exemplary implant system of FIG. 16, in accordance with the present disclosure;

FIG. 20 is a front view of an exemplary implant of the exemplary implant system of FIG. 16, in accordance with the present disclosure;

FIG. 21 is a front perspective view of an exemplary implant of the exemplary implant system of FIG. 16, in accordance with the present disclosure;

FIG. 22 is a rear perspective view of an exemplary implant of the exemplary implant system of FIG. 16, in accordance with the present disclosure;

FIG. 23 is a top, front perspective view of an exemplary orthopedic system, in accordance with the present disclosure;

FIG. 24 is a top, rear perspective view of the exemplary orthopedic system of FIG. 23, in accordance with the present disclosure;

FIG. 25 is a top view of the exemplary orthopedic system of FIG. 23, in accordance with the present disclosure;

FIG. 26 is a front view of the exemplary orthopedic system of FIG. 23, in accordance with the present disclosure;

FIG. 27 is a side view of the exemplary orthopedic system of FIG. 23, in accordance with the present disclosure;

FIG. 28 is a rear view of the exemplary orthopedic system of FIG. 23, in accordance with the present disclosure;

FIG. 29 is an alternate side view of the exemplary orthopedic system of FIG. 23, in accordance with the present disclosure;

FIG. 30 is an exploded view of the exemplary orthopedic system of FIG. 23, in accordance with the present disclosure;

FIG. 31 is a top view of the exemplary orthopedic system of FIG. 23 shown with the angel wing attached to a foot of a patient, in accordance with the present disclosure;

FIG. 32 is a top view of the exemplary orthopedic system of FIG. 23 shown adjacent to the foot of the patient, in accordance with the present disclosure;

FIG. 33 is a medial side view of the exemplary orthopedic system of FIG. 23 shown adjacent to the foot of the patient, in accordance with the present disclosure;

FIG. 34 is a top, front view of the exemplary orthopedic system of FIG. 23 shown adjacent to the foot of the patient, in accordance with the present disclosure;

FIG. 35 is a top view of a portion of the exemplary orthopedic system of FIG. 23 shown adjacent to the foot of the patient, in accordance with the present disclosure;

FIG. 36 is a top, rear view of the exemplary orthopedic system of FIG. 23 shown adjacent to the foot of the patient, in accordance with the present disclosure;

FIG. 37 is a top, rear view of a portion of the exemplary orthopedic system of FIG. 23 shown adjacent to the foot of the patient, in accordance with the present disclosure;

FIG. 38 is top view of the exemplary orthopedic system of FIG. 23 showing a step of an exemplary surgical method, in accordance with the present disclosure;

FIG. 39 is a front view of the exemplary orthopedic system of FIG. 23 showing a step of an exemplary surgical method, in accordance with the present disclosure;

FIG. 40 is a top view of the exemplary orthopedic system of FIG. 23 showing a step of an exemplary surgical method, in accordance with the present disclosure;

FIG. 41 is a front view of the exemplary orthopedic system of FIG. 23 showing a step of an exemplary surgical method, in accordance with the present disclosure;

FIG. 42 is a top view of the exemplary orthopedic system of FIG. 23 showing a step of an exemplary surgical method, in accordance with the present disclosure;

FIG. 43 is a top view of the exemplary orthopedic system of FIG. 23 showing a step of an exemplary surgical method, in accordance with the present disclosure;

FIG. 44 is a medial side view of the exemplary orthopedic system of FIG. 23 showing a step of an exemplary surgical method, in accordance with the present disclosure;

FIG. 45 is a top view of the exemplary orthopedic system of FIG. 23 showing a step of an exemplary surgical method, in accordance with the present disclosure; and

FIG. 46 is a top view of the exemplary orthopedic implant system of FIG. 21, in accordance with the present disclosure.

### DETAILED DESCRIPTION OF THE INVENTION

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

The instruments, implants, systems, assemblies, and related methods for maintaining, correcting, and/or resurfacing joint surfaces of the present disclosure may be similar to, such as include at least one feature or aspect of, the implants, systems, assemblies and related methods disclosed in International PCT Application No. PCT/US2018/20046, filed on Feb. 27, 2018, and entitled Intramedullary Nail Alignment Guides, Fixation Guides, Devices, Systems, and Methods of Use; International PCT Application No. PCT/US2018/64368, filed on Dec. 17, 2018, and entitled Alignment Guides, Cut Guides, Systems and Methods of Use and Assembly; International PCT Application No. PCT/US2019/041146, filed on Jul. 10, 2019, and entitled Guides, Instruments, Systems and Methods of Use; and/or International PCT Application No. PCT/US2014/27086, filed on Mar. 14, 2014, and entitled Intramedullary Nail Fixation Guides, Devices, and Methods of Use; and/or U.S. Pat. No. 9,980,760 filed on Nov. 19, 2014, and entitled Step Off Bone Plates, Systems, and Methods of Use; and/or U.S. Patent No. D720,456 filed on Jul. 26, 2012 and entitled Lapidus Bone Wedge; and/or U.S. Patent No. D765,844 filed on Oct. 23, 2014 and entitled Bone Plate; and/or U.S. Patent No. D695,402 filed on Dec. 10, 2013 and entitled Lapidus Cut Guide; and/or U.S. Patent No. D904,2016 filed on Nov. 22, 2017 and entitled Intramedullary Fastener; and/or U.S. Patent No. D865,173 filed on Jul. 9, 2018 and entitled Cut Guide; and/or U.S. patent application Ser. No. 29/686,941 filed on Apr. 9, 2019 and entitled Cut Guide; and/or U.S. Patent No. D904,609 filed on Apr. 9, 2019 and entitled Cut Guide; and/or U.S. Patent No. D9042010 filed on Apr. 9, 2019 and entitled Cut Guide; which are hereby incorporated herein by reference in their entireties. Similarly, the instruments, implants, systems, assemblies, and related methods for maintaining, correcting, and/or resurfacing joint surfaces of the present disclosure may include one or more instrument (e.g., one or more insertion and/or implantation instruments) disclosed in U.S. Provisional Application No. 63/173,043, filed Apr. 9, 2021 and entitled Surgical Instruments, Guides, and Methods of Use; and/or International PCT Application No. PCT/US2018/20046, filed on Feb. 27, 2018, and entitled Intramedullary Nail Alignment Guides, Fixation Guides, Devices, Systems, and Methods of Use; and/or International PCT Application No. PCT/US2018/64368, filed on Dec. 17, 2018, and entitled Alignment Guides, Cut Guides, Systems and Methods of Use and Assembly; and/or International PCT Application No. PCT/US2019/041146, filed on Jul. 10, 2019, and entitled Guides, Instruments, Systems and Methods of Use; and/or International PCT Application No. PCT/US2014/27086, filed on Mar. 14, 2014, and entitled Intramedullary Nail Fixation Guides, Devices, and Methods of Use; and/or U.S. Pat. No. 9,980,760 filed on Nov. 19, 2014, and entitled Step Off Bone Plates, Systems, and Methods of Use; and/or U.S. Patent No. D720,456 filed on Jul. 26, 2012 and entitled Lapidus Bone Wedge; and/or U.S. Patent No. D765,844 filed on Oct. 23, 2014 and entitled Bone Plate; and/or U.S. Patent No. D695,402 filed on Dec. 10, 2013 and entitled Lapidus Cut Guide; and/or U.S. Patent No. D904,2016 filed on Nov. 22, 2017 and entitled Intramedullary Fastener; and/or U.S. Patent No. D865,173 filed on Jul. 9, 2018 and entitled Cut Guide ; and/or U.S. patent application Ser. No. 29/686,941 filed on Apr. 9, 2019 and entitled Cut Guide; and/or U.S. Patent No. D904,609 filed on Apr. 9, 2019 and entitled Cut Guide; and/or U.S. Patent No. D9042010 filed on Apr. 9, 2019 and entitled Cut Guide; and/or U.S. Provisional Ser. No. 63/262,845 filed on Oct. 21, 2021 and entitled Surgical Instruments, Guides, and Methods of Use; and/or U.S. Provisional Ser. No. 63/304,144 filed on Jan. 28, 2022 and entitled Surgical Instruments, Guides, and Methods of Use; and/or U.S. Provisional Ser. No. 63/484,092 filed on Feb. 9, 2023 and entitled Lapidus Clamp and Methods of Use; which are hereby incorporated herein by reference in their entireties.

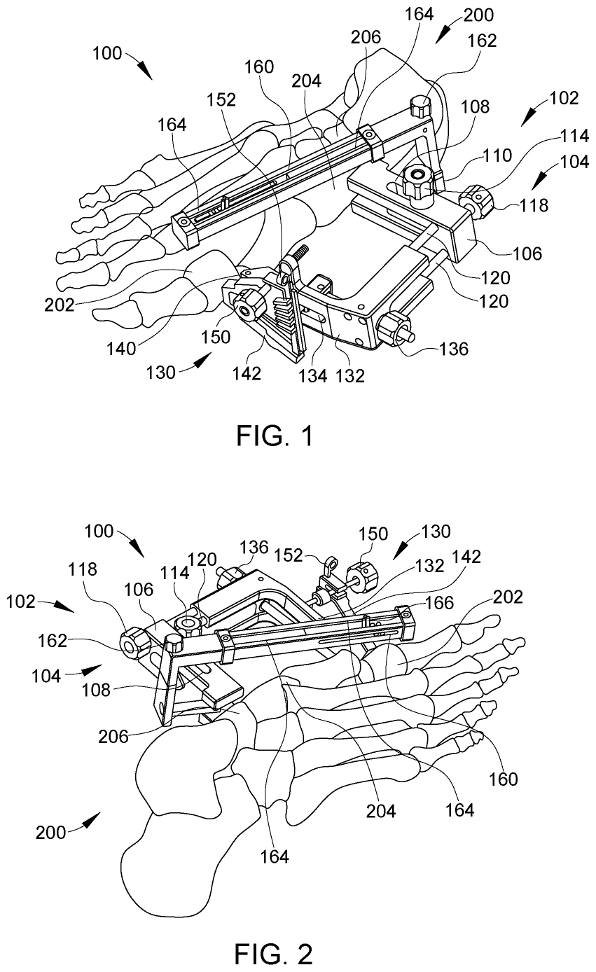

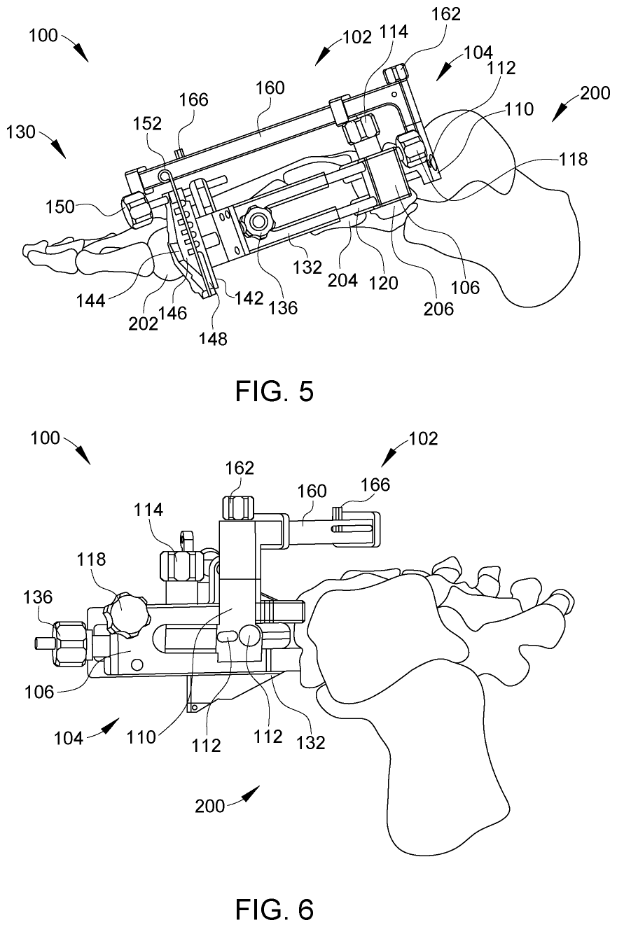

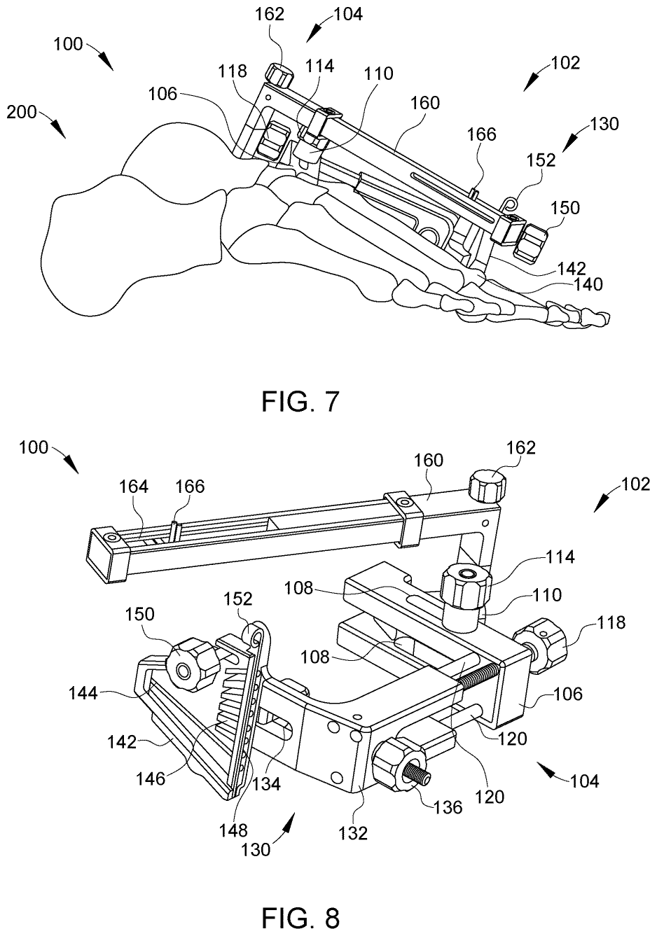

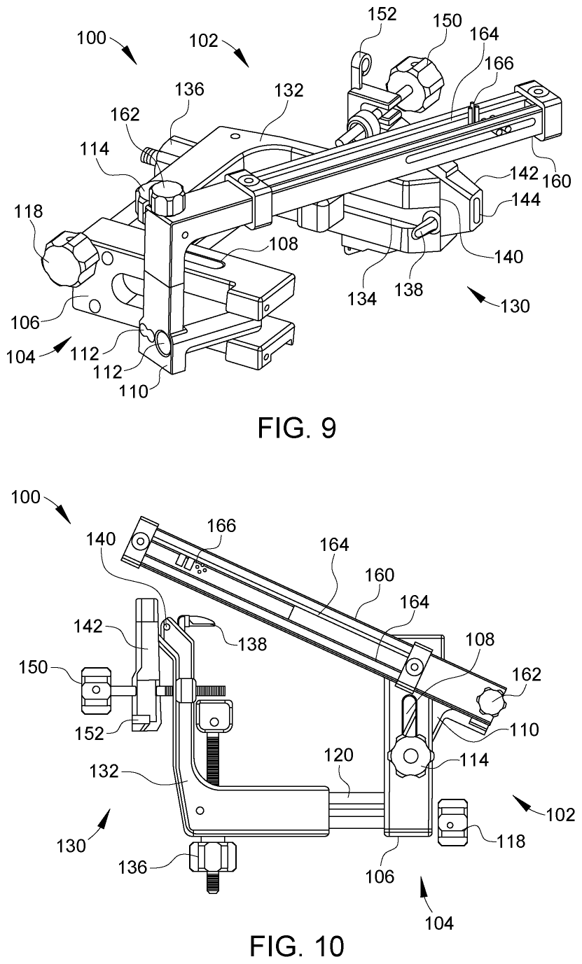

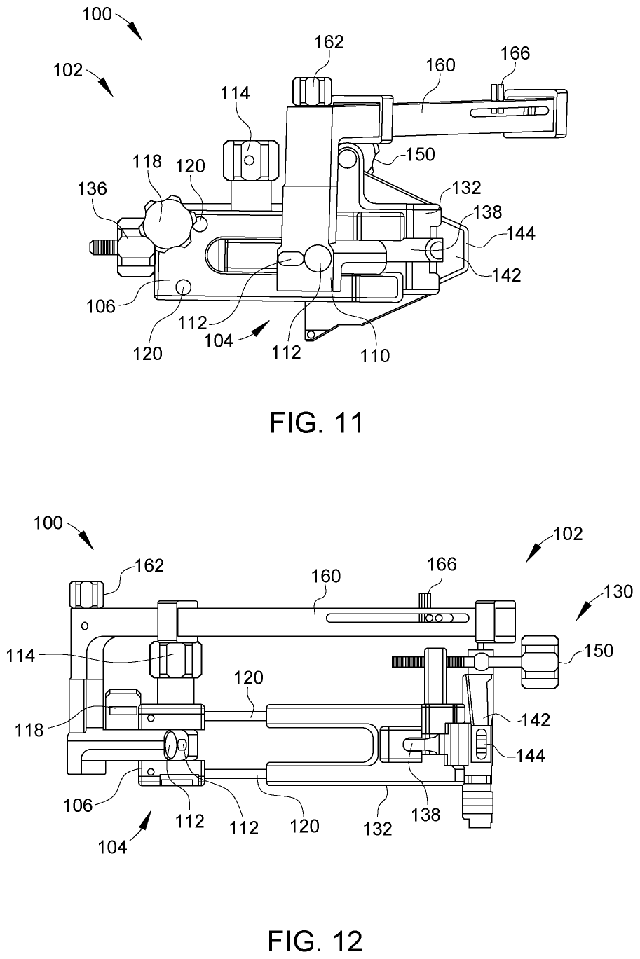

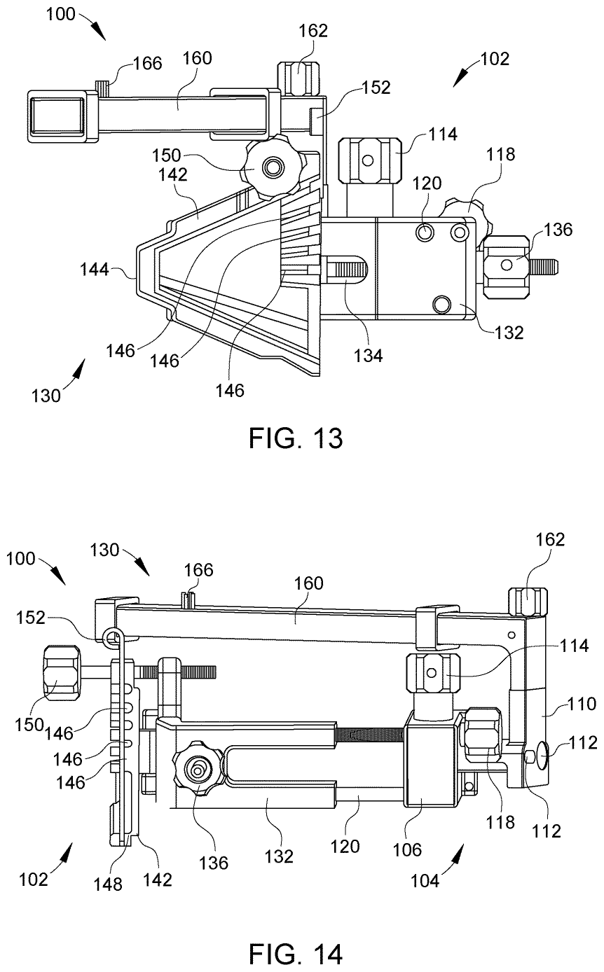

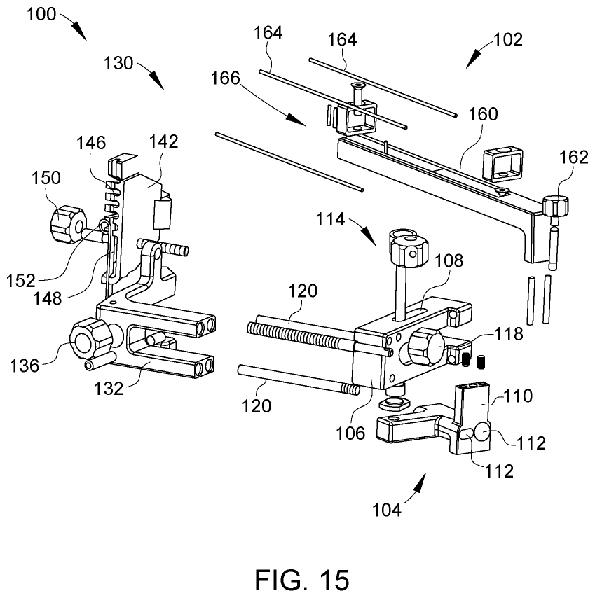

Referring now to FIGS. 1-15, an orthopedic system 100 (referred to hereinafter as “system 100”) for correcting bunion deformities is shown (and shown adjacent to the anatomy of the foot with reference to FIGS. 1-8), according to an exemplary embodiment. In some aspects, the system 100 may be implemented in conjunction with one or more other instruments, implants, or surgical methods common to bunion correction procedures including but not limited to those incorporated by reference herein. For example, the system 100 may be implemented in conjunction with one or more cut guides configured to guide one or more cuts to the first metatarsal and/or medial cuneiform of a patient, with said system subsequently implemented to guide correction and fixation of the first metatarsal and/or medial cuneiform. The system 100 may also be implemented in conjunction with various instrumentation common to orthopedic procedures, including but not limited to powered instrumentation such as sagittal/reciprocating saws and powered drivers. In some aspects, one or more components of the system 100 may be implemented in conjunction with one or more components of other orthopedics systems, and/or one or more components of other orthopedic systems may be implemented in conjunction with the system 100.

The system 100 is shown to include an instrument 102 which may be coupled with at least one of a first metatarsal 202 (e.g., a distal portion thereof) and a navicular 206 of a foot 200 of a patient, with at least a portion of the instrument 202 positioned adjacent to a medial cuneiform 204 of the foot 200. In some aspects, the instrument 102 may be coupled with the first metatarsal 202 via one or more k-wires inserted in and received at least partially therethrough openings in the instrument and into the aforementioned bony structures. For example, the instrument 102 may be releasably coupled with the navicular 206 via one or more k-wires, and may also be coupled with at least a portion of the first metatarsal 202 (e.g., a distal segment separated from a proximal segment via an osteotomy cut). The instrument 102 is shown to include a proximal portion 104 and a distal portion 130 (which may be integral or releasably couplable with one another), where the proximal portion 104 is coupled with the navicular 206 and the distal portion 130 is coupled with the first metatarsal 202. In some aspects, the instrument 102 (and components thereof) may couple with additional and/or alternate anatomical structures, for example the medial cuneiform 204 and/or adjacent/lesser metatarsals.

The proximal portion 104 is shown to include a body 106 having a substantially u-shaped geometry which, as shown in at least FIG. 1, may be positioned in practice such that a first leg of the u-shape is positioned at least partially above a second leg of the u-shape. As shown, each of the legs of the u-shape of the body 106 include an opening 108 extending from a top surface therethrough to a bottom surface, which are shown in the exemplary embodiment of FIGS. 1-15 as elongated openings. In some aspects the openings 108 may include alternate geometries, for example circular openings. The proximal portion 104 is further shown to include a targeting guide 110 (referred to hereinafter as “guide 110”), which is positioned at least partially between the two legs of the u-shape of the body 106 and includes protrusions extending in opposite directions from the top and bottom surfaces of the guide 110 which are configured to be received within each of the openings 108. As shown, the protrusions of the guide 110 each include a geometry complimentary to the height of the openings 108 and a width lesser than that of the openings 108. Accordingly, the guide 110 is translatably coupled (and thus translatable relative to) with the body 106, with the guide 110 translatable along the length of the openings 108.

The guide 110 is shown to include a pair of openings 112 extending from a first side surface through the guide to a second side surface, according to the exemplary embodiment of FIGS. 1-15. In some aspects, the guide 112 may include a single opening 112 or may include three or more openings 112. In some aspects, one or more of the openings 112 may be a scalloped opening so as to facilitate placement of components therethrough (e.g., a drill sleeve) at variable distances from the opposite opening 112. As shown, the openings 112 are positioned adjacent one another laterally and extend along axes parallel to one another. In some aspects, the openings 112 may extend along axes that are converging or diverging. Further, in some aspects the guide 110 may be decouplable from the body 106 such that a guide 110 having openings with alternate trajectories may be coupled with the body 106 (e.g., if the first guide 110 has openings 112 with parallel trajectories, a physician may swap the first guide 110 for a second guide 110 that has openings 112 with converging or diverging trajectories).

The guide 110 is further shown to include a vertical cannulation extending therethrough and establishing fluid communication through the guide 110 along the cannulation from one protrusion (e.g., the upper protrusion) to the other (e.g., the lower protrusion). The vertical cannulation may extend along an axis substantially perpendicular to one or more axes of the openings 112 of the guide 110. The cannulation of the guide 110 is shown to receive at least a portion of an actuator 114 therethrough such that a knob of the actuator 114 abuts the upper protrusion of the guide 110 and a terminal end of the actuator 114 abuts the lower protrusion of the guide 110. Manipulation of the actuator 114 (e.g., by rotating the knob thereof) permits or limits translation of the guide 110 within the range of motion defined by the boundaries of the openings 108. Additionally, the guide 110 is rotatable about the axis of the actuator 114, so manipulation (e.g., tightening) of the actuator 114 retains the guide 110 is a desired rotational position relative to the actuator 114 and a desired translatable position relative to the openings 108.

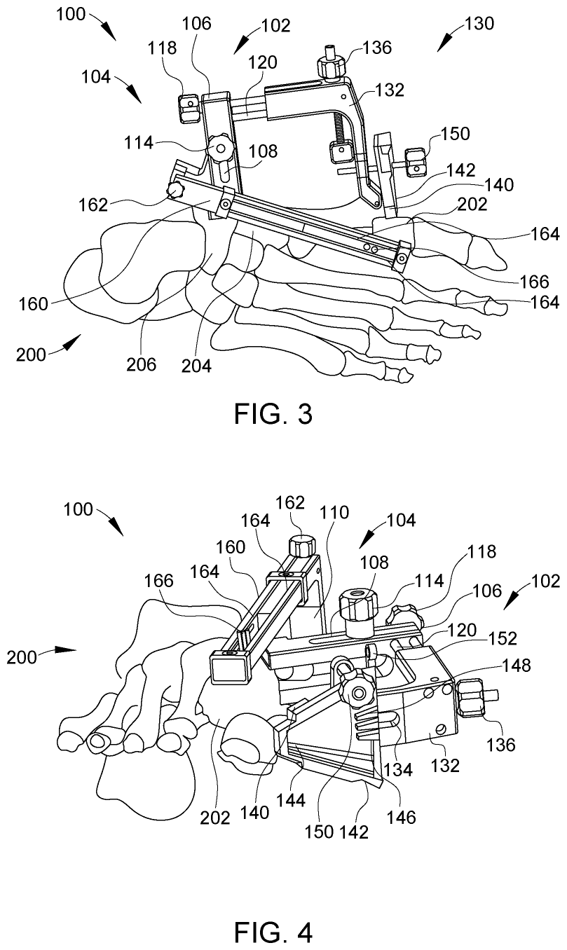

The proximal portion 104 of the instrument 102 is further shown to include an angel wing 160, with the angel wing 160 releasably couplable with the guide 110. Accordingly, any translation and/or rotation of the guide 110 is also applied to the angel wing 160. The angel wing 160, as shown, includes a substantially elongated (e.g., rectangular) geometry, with a coupling portion 162 compatible with the guide 110 positioned at a first end thereof. The coupling portion 162 may include a threaded actuator or other instrument configured to facilitate coupling with complementary components of the guide 110 disposed in an extension extending upward from a portion of the guide 110. The angel wing 160 is further shown to include at least one indicator 164, as seen in FIGS. 1-15 as a pair of linear radiopaque rods positioned parallel to one another, positioned along a length thereof between the first end and a terminal second end of the angel wing 160. In the coupled configuration as shown in at least FIG. 3, the axes/trajectories of each of the indicators is parallel to a corresponding axis/trajectory of each of the openings 112, with the axes/trajectories of the openings 112 positioned directly below that of the indicators 162. Accordingly, the trajectory of the openings 112 can be viewed over the first metatarsal 202, the medial cuneiform 204, the navicular 206, etc. under fluoroscopy based on the radiopaque property of the indicators 164. The angel wing 160 is further shown to include indicators 166 positioned between the indicators 164 and the terminal end of the angel wing 160. In the exemplary embodiment shown in FIGS. 1-15, the indicators 166 are seen as four short, vertically-positioned radiopaque rods and a pair of horizontally-positioned short, radiopaque rods positioned distal to the four aforementioned rods. The four vertically positioned rods, when viewed on fluoroscopy, will appear as four equidistantly-spaced dots when viewed from a perfectly superior angle, which allows a physician to ensure proper angulation of the instrument 102 under fluoroscopy. The two horizontally positioned rods, when viewed under fluoroscopy, will appear as two lines perpendicular to the indicators 164 and, similar to the four vertically positioned rods, are indicative of any angulation in the positioning of the instrument 102 under fluoroscopy. Accordingly, a physician may manipulate the guide 110 and/or other components of the instrument 102 so as to adject the trajectories of the openings 112 based on the indications of the trajectories and the positioning of the instrument 102 based on the indicators 164, 166 of the angel wing 160.

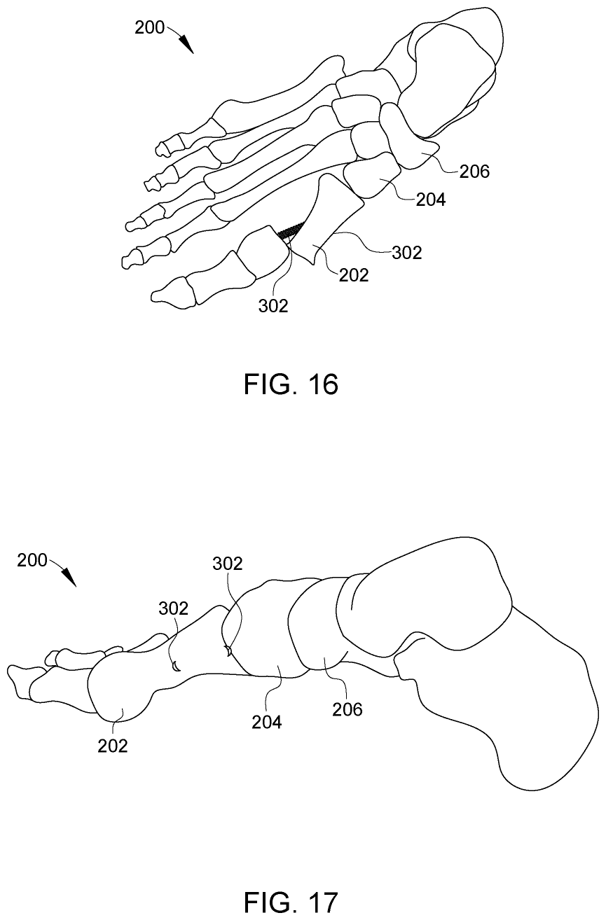

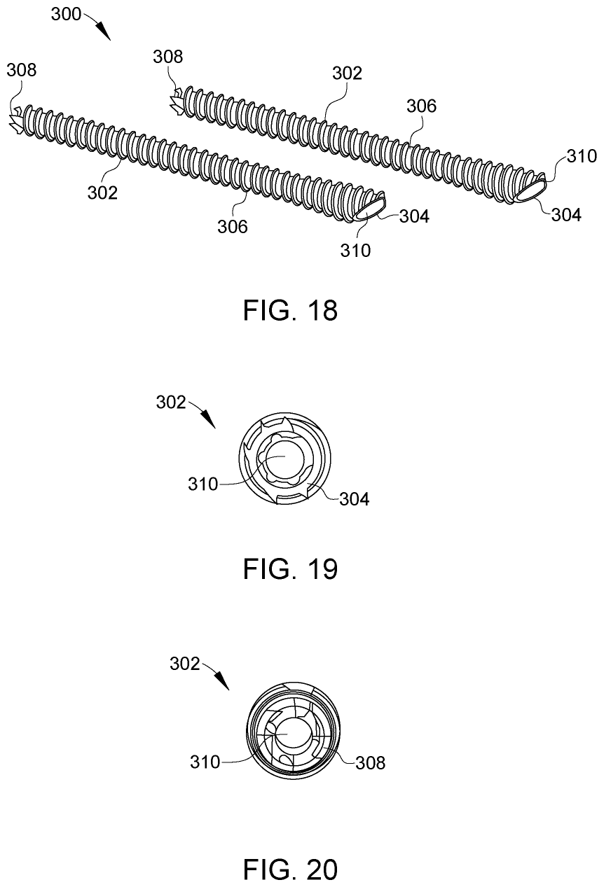

In implementing the instrument 102, a physician may position the guide 110 in a desired rotational and translational position (based on visual feedback from the indicators 164, 166), and manipulate the actuator 114 so as to retain the guide 110 in such a desired position. The physician may then implement one or more instruments, for example sleeves/guides for drills, k-wires, or other instruments, to place a k-wire through one of the openings 112 and into both a first and second segment of the first metatarsal 202 (where the first metatarsal 202 as already been osteotomized at a point along the length thereof, as shown in FIGS. 1-8). Once the k-wire has been placed, the physician may then place a cannulated drill bit over the k-wire and drill along the trajectory of the k-wire so as to guide the path of the drill bit and prevent skiving. After a pilot hole has been created by the drill bit, the physician may place an implant 302, for example from an implant system 300 such as that shown in FIGS. 16-22, into the pilot hole, thus providing fixation of the proximal and distal segments of the first metatarsal 202. In some aspects, the k-wire may be removed prior to the placement of the implant 302. In some aspects, the lateral-most opening of the openings 112 may be drilled and the implant 302 placed therethrough prior to the lateral-most opening 112 being drilled and the implant 302 placed therethrough. In some aspects, a physician may adjust the trajectory of the openings 112 by manipulating the guide 110 and actuator 114 after a first implant 302 is placed, but prior to a second implant 302 being placed. For example, a first implant 302 may be placed and, in the instance the physician desires converging or diverging trajectories for the two implants 302, the guide 110 may be rotated and/or translated such that the trajectory of the second opening 112 (e.g., the medial-most opening 112) is oblique relative to an axis of the already-placed implant 302. The physician may then repeat the steps of placing a k-wire through the opening 112 (now the medial-most opening 112), guiding a drill bit over the k-wire, and finally placing an implant 302 within the pilot hole created by the drill bit such that an axis of the implant 302, once placed, is oblique relative to that of the first-placed implant 302. It should be understood that, in some aspects, the implants 302 may be placed with axes thereof parallel to one another (e.g., the guide 110 is not adjusted between placement of the first and second implants 302). Further, it should be understood that the aforementioned steps may be performed in an alternate order. For example, the medial-most opening 112 and implant 302 may be drilled and implanted, respectively, prior to the lateral-most opening 112 and implant 302. Further, both openings 112 may be drilled prior to the implantation of any implants 302.

The distal portion 130 of the instrument 102 is shown to include a body 132 having a substantially L-shaped geometry, where each leg of the L-shape includes a substantial u-shape (e.g., rotated 90-degrees) similar to that of the body 106. The proximal-most leg of the L-shape is shown to be slidably coupled with the body 106 via a pair of rails 120 extending parallel to one another with at least one of the proximal and distal portions 104, 130 slidably coupled with the other. The body 132 is further shown to receive at least a portion of an actuator 118 positioned adjacent the body 106 and include a threaded shaft extending from a knob portion. The threaded shaft is shown to extend through a portion of the body 106 and be at least partially received by a portion of the body 132. Accordingly, manipulation of the actuator 118 may translate either the proximal portion 104 or the distal portion 130 toward the other (along the rails 120) as the threaded rod portion of the actuator 118 advances within or withdraws from the threading of the respective opening of the body 132.

The body 132 is further shown to include a pair of openings 134 disposed in the legs of the L-shape of the body 132, with the openings 134 being the open portion of the u-shapes of the legs of the body 132. The body 132 is shown to include a bore positioned distal relative to the proximal opening 134 which includes an actuator 136 extending therethrough. The actuator 136 is shown to include a knob portion on a medial-most side of the body 132 and a threaded portion extending through the bore to the lateral side of the body 132. The threaded portion of the actuator 136 may be translated in the medial-lateral direction by rotating the knob portion of the actuator 136. The actuator 136 is shown to be coupled (but may be integral in some aspects) with an engagement member 138 at a distal end of the threaded portion of the actuator 136. The engagement member 138 includes an opening configured to receive at least a portion of the threaded portion of the actuator 136 therein. Accordingly, translation of the threaded portion of the actuator 136 as a result of manipulation of the knob portion of the actuator 136 similarly translates the engagement member 138.

The engagement member 138 is shown to include a protrusion extending therefrom in at least one of the proximal and/or lateral directions, shown in FIGS. 1-15 as a hooked member. The hooked member of the engagement member 138 may be positioned adjacent an osteotomy site of the first metatarsal 202, with the distal-most portion of the hooked member positioned within the intramedullary canal of the proximal segment of the first metatarsal 202 such that at least a portion of the hooked member abuts a wall of the intramedullary canal. Accordingly, the actuator 136 may then be manipulated so as to reposition the proximal segment of the first metatarsal 202 relative to the distal segment of the first metatarsal 202.

The distal portion 130 is further shown to include an adjustment mechanism 142 which is releasably and pivotably coupled with the body 132 via a coupling 140. The coupling 140 is positioned at the terminal end of the opening 134 of the distal-most leg of the L-shape of the body 132 such that the adjustment mechanism 142 is rotatable (e.g., pivotable) about the coupling 140. The adjustment mechanism 142 is also releasably and translatably coupled with the body 132 via an actuator 136 which includes a knob portion and a threaded portion, with at least a portion of the threaded portion received through a threaded opening of the body 132 (e.g., positioned on a portion of the distal-most leg of the L-shape). Accordingly, manipulation of the knob portion of the actuator 136 drives translation of the adjustment mechanism 142 closer/further in the proximal-distal direction relative to the body 132.

The adjustment mechanism 142 is shown to include an opening 144 at a first end, shown in FIG. 4 as a lateral-most end, and an opening 148 opposite the adjustment mechanism 142 at a second end (shown in FIG. 4 as a medial-most end). The adjustment mechanism 142 further includes one or more slots 146 having an elongated geometry and positioned between the openings 144 and 148. Accordingly, a k-wire may be coupled with the first metatarsal 202 (e.g., a distal segment thereof) and extend through the opening 144, into the adjustment mechanism 144, through one of the slots 146, and out the adjustment mechanism 142 via the opening 148. Further the k-wire may be manipulated by a physician from one of the lower slots 146 to a higher slot 146 so as to correct a rotational deformity of the first metatarsal 202 (and/or a segment thereof). In some aspects, the slots 146 may be labeled, for example with specific angular measurements, to quantify the correction achieved with manipulation of the k-wire from one of the slots 146 to the next. The adjustment mechanism 142 further includes a locking mechanism 152 positioned adjacent the opening 148 configured to retain the k-wire in a desired slot 146 after placement, as the slots 146 are open on one side so as to permit placement therein of the k-wire. The locking mechanism 152 is shown to be pivotably coupled with the adjustment mechanism 142 adjacent the bottom-most portion of the opening 148, and may be rotated (e.g., pivoted about the coupling point) to releasably couple with a point adjacent the upper-most portion of the opening 148 so as the k-wire in the desired slot 146.

In some aspects, a physician may manipulate the distal segment of the first metatarsal 202 via the adjustment mechanism 142 (and a k-wire) and the proximal segment of the first metatarsal 202 via the engagement member 138 to achieve a desired position of the segments. The physician may then manipulate the actuator 136 and/or the locking mechanism 152 to retain the aforementioned segments in the desired position prior to manipulating any components of the proximal portion 104 of the instrument 102 as described previously herein.

Referring now to FIGS. 16-22, the implant system 300 and implants 302 thereof are shown, according to an exemplary embodiment. The implants 302 of the system 300 may be implemented in conjunction with the system 100 as described with reference to the system 100 and the instrument 102. In some aspects, the system 300 and the implants 302 may be implemented in conjunction with additional or alternate instrumentation to that of the system 300 and, further, in some aspects may be implemented in various anatomy including but not limited to that of the foot and ankle. In some aspects, the system 300 may include two implants 302 as shown in FIGS. 16-18, and in some aspects, the system 300 may include three or more implants 302. Further, in some aspects the system 300 may include a single implant 302.

As shown in FIGS. 16-17, a pair of implants 302 have been placed in the first metatarsal 202 of a patient so as to provide fixation (and ultimately facilitate fusion) of two bony segments of the first metatarsal 202. As seen, the implants 302 are positioned substantially parallel to one another, but in some aspects may be positioned at an oblique angle relative to one another (e.g., converging or diverging). In implanting the implants 302, the implants 302 may be inserted first into a proximal segment of the first metatarsal 202 and then into a distal segment of the first metatarsal 202, with the proximal and distal segments positioned opposite an osteotomy cut made to a portion of the shaft of the first metatarsal 202.

The implant 302 (which, in FIGS. 16-18, are seen to be adjacent to one another) are shown to include a head portion 304 opposite a shaft portion 306 from a tip portion 308, according to an exemplary embodiment. The shaft 306 is shown to include a threading extending along at least a portion thereof, which is shown in an exemplary embodiment to extend substantially from the head portion 304 to the tip 308. In some aspects, the shaft 306 may include one or more openings along the length thereof, for example vented portions, which may interrupt the threading for a portion of the length of the shaft 306.

The head portion 304 is shown to include a chamfered surface (e.g., and angled surface) configured such that when the implant 302 is positioned in a certain phase (e.g., degree of rotation) relative to an adjacent surface, the chamfer surface sits substantially flush with the adjacent surface (e.g., at least a portion of the chamfer surface is positioned in the same or an adjacent plane to that of the adjacent surface). The head portion 304 is also shown to include a drive feature (which may be a standard drive feature) recessed relative to the chamfer surface of the head portion 304. As shown, at least a portion of the drive feature is positioned (e.g., recessed) below the plane of the chamfer surface.

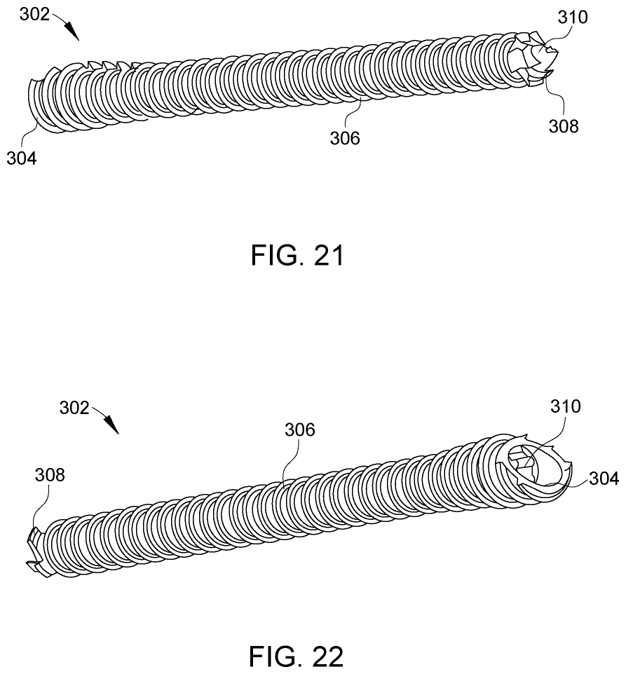

The tip 308 of the implant 302 may include at least one cutting flute (which may have at least one respective relief area) positioned at the terminal end of the tip 308. As shown in at least FIG. 21, the tip 308 includes multiple cutting flutes (e.g., four cutting flutes) and respective relief areas. In some aspects, the implant 302 may be self-tapping (e.g., does not require a pilot hole to be drilled prior to implantation). However, in some aspects a surgical method may recommend or require a pilot hole be drilled prior to implantation of the implant 302 regardless of whether the implant 302 includes self-tapping features.

The implant 302 is further shown to include a cannulation 310 extending along a length thereof and therethrough from the head 304 to the tip 308. The openings disposed in the outer surface of the shaft 306 may extend through at least a portion of the shaft 306 so as to enable fluid communication between an inner portion of the implant 302 (e.g., the cannulation 310 and an outer surface of the shaft 306). In some aspects, the cannulation 310 may include a substantially cylindrical volume, where a longitudinal axis of the cannulation 310 extends along a trajectory forming an oblique angle with the plane of the chamfer surface at the head portion 304. In some aspects, the implant 302 may include a taper along a length thereof. As seen in at least FIG. 21, the implant 302 has a greater lateral dimension adjacent the head portion 304 than adjacent the tip 308, with the lateral dimension of the implant 302 gradually decreasing along at least a portion of the length of the shaft 306.

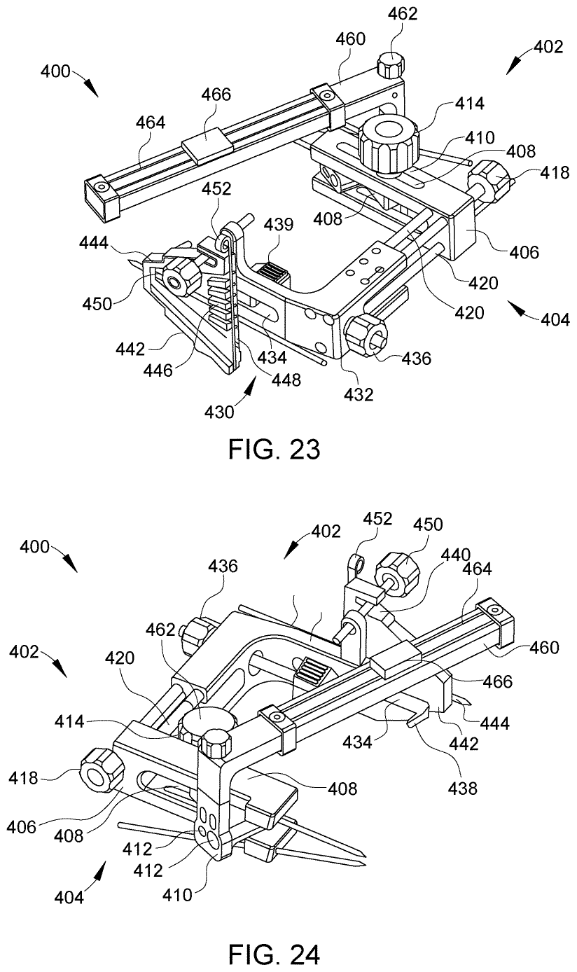

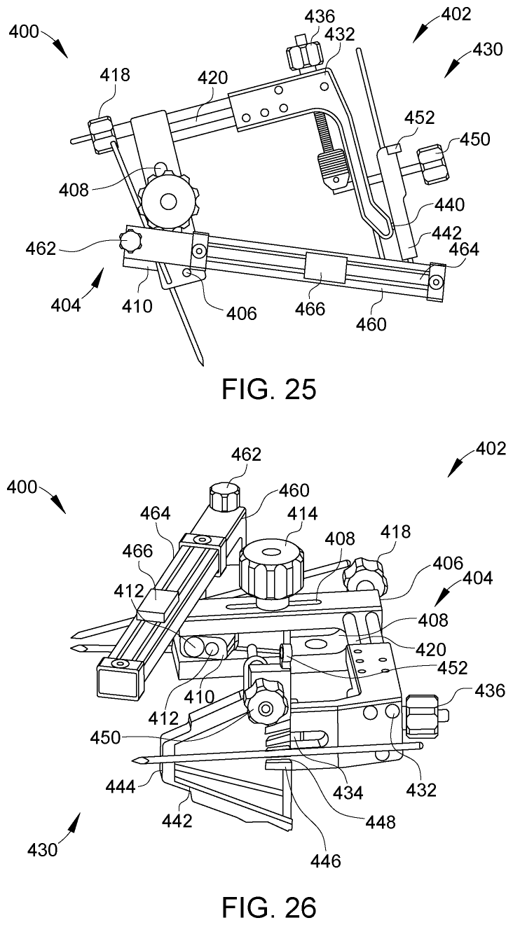

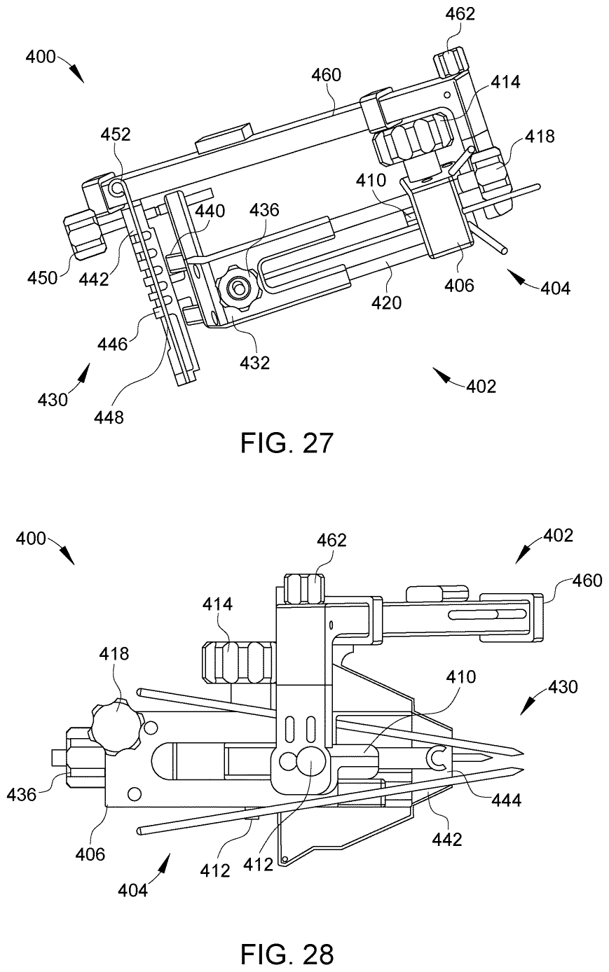

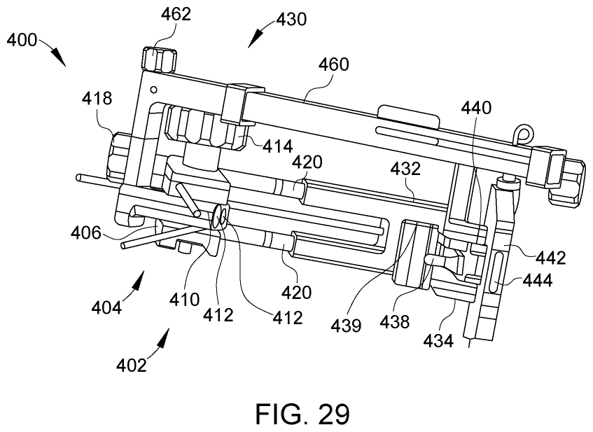

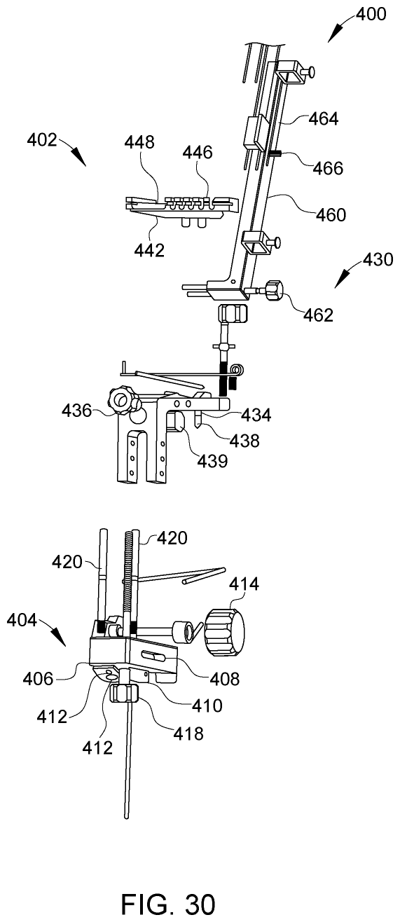

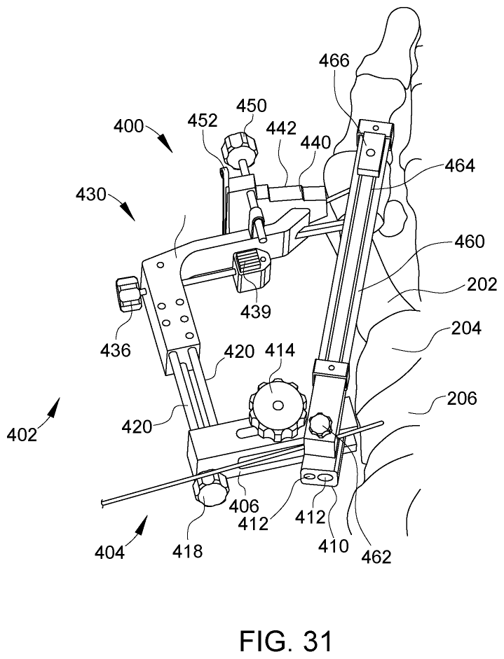

Referring now to FIGS. 23-37, an orthopedic system 400 (referred to hereinafter as “system 400”) for correcting bunion deformities is shown (and shown adjacent to the anatomy of the foot with reference to FIGS. 31-37), according to an exemplary embodiment. In some aspects, the system 400 may be implemented in conjunction with one or more other instruments, implants, or surgical methods common to bunion correction procedures including but not limited to those incorporated by reference herein (e.g., the system 100 and components thereof, as well one or more implants 302). For example, the system 400 may be implemented in conjunction with one or more cut guides configured to guide one or more cuts to the first metatarsal 202 and/or medial cuneiform 204 of a patient, with the system subsequently implemented to guide correction and fixation of the first metatarsal and/or medial cuneiform. The system 400 may also be implemented in conjunction with various instrumentation common to orthopedic procedures, including but not limited to powered instrumentation such as sagittal/reciprocating saws and powered drivers. In some aspects, one or more components of the system 400 may be implemented in conjunction with one or more components of other orthopedics systems, and/or one or more components of other orthopedic systems may be implemented in conjunction with the system 400.

The system 400 is shown to include an instrument 402 which may be coupled with at least one of a first metatarsal 202 (e.g., a distal portion thereof) and a navicular 206 of the foot 200 of a patient, with at least a portion of the instrument 402 positioned adjacent a medial cuneiform 204 of the foot 200. In some aspects, the instrument 402 may be coupled with the first metatarsal 202 via one or more k-wires inserted in and received at least partially therethrough openings in the instrument and into the aforementioned bony structures. For example, the instrument 402 may be releasably coupled with the navicular 206 via one or more k-wires, and may also be coupled with at least a portion of the first metatarsal 202 (e.g., a distal segment separated from a proximal segment via an osteotomy cut). The instrument 402 is shown to include a proximal portion 404 and a distal portion 430 (which may be integral or releasably couplable with one another), where the proximal portion 404 is coupled with the navicular 206 and the distal portion 430 is coupled with the first metatarsal 202. In some aspects, the instrument 402 (and components thereof) may couple with additional and/or alternate anatomical structures, for example the medial cuneiform 204 and/or adjacent/lesser metatarsals.

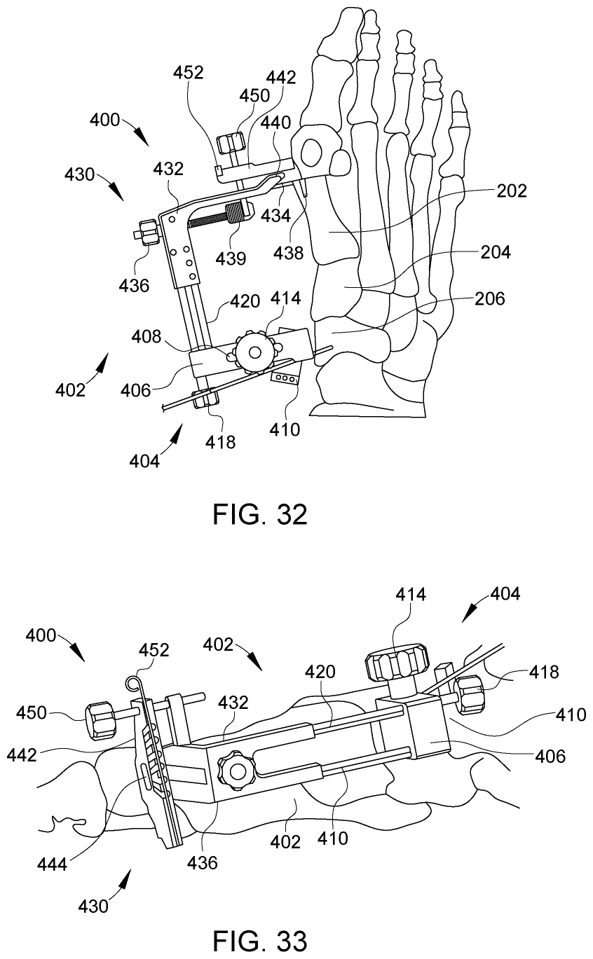

The proximal portion 404 is shown to include a body 406 having a substantially u-shaped geometry which, as shown in at least FIG. 23, may be positioned in practice such that a first leg of the u-shape is positioned at least partially above a second leg of the u-shape. As shown, each of the legs of the u-shape of the body 406 include an opening 408 extending from a top surface therethrough to a bottom surface, which are shown in the exemplary embodiment of at least FIGS. 23 and 32 as elongated openings. In some aspects, the openings 408 may include alternate geometries, for example circular openings. The proximal portion 404 is further shown to include a targeting guide 410 (referred to hereinafter as “guide 410”), which is positioned at least partially between the two legs of the u-shape of the body 406 and includes protrusions extending in opposite directions from the top and bottom surfaces of the guide 410 which are configured to be received within each of the openings 408. As shown, the protrusions of the guide 410 each include a geometry complimentary to the height of the openings 408 and a width lesser than that of the openings 408. Accordingly, the guide 410 is translatably coupled (and thus translatable relative to) with the body 406, with the guide 410 translatable along the length of the openings 408.

The guide 410 is shown to include a pair of openings 412 extending from a first side surface through the guide 410 to a second side surface, according to the exemplary embodiment shown in FIGS. 24 and 36-37. In some aspects, the guide 410 may include a single opening 412 or may include three or more openings 412. In some aspects, one or more of the openings 412 may be a scalloped opening so as to facilitate placement of components therethrough (e.g., a drill sleeve) at variable distances from the opposite opening 412. As shown, the openings 412 are positioned adjacent to one another laterally and extend along axes parallel to one another. In some aspects, the openings may extend along axes that are converging or diverging. Further, in some aspects, the guide 410 may be decouplable from the body 406 such that a guide 410 having openings with alternate trajectories may be coupled with the body 406 (e.g., if the first guide 410 has openings 412 with parallel trajectories, a physician may swap the first guide 410 for a second guide 410 that has openings 412 with converging or diverging trajectories).

The guide 410 is further shown to include a vertical cannulation extending therethrough and establishing fluid communication through the guide 410 along the cannulation from one protrusion (e.g., the upper protrusion) to the other (e.g., the lower protrusion). The vertical cannulation may extend along an axis substantially perpendicular to one or more axes of the openings 412 of the guide 410. The cannulation of the guide 410 is shown to receive at least a portion of an actuator 414 therethrough such that a knob of the actuator 414 abuts the upper protrusion of the guide 410 and a terminal end of the actuator 414 abuts the lower protrusion of the guide 410. Manipulation of the actuator 414 (e.g., by rotating the knob thereof) permits or limits translation of the guide 410 within the range of motion defined by the boundaries of the openings 408. Additionally, the guide 410 is rotatable about the axis of the actuator 414, so manipulation (e.g., tightening) of the actuator 414 retains the guide 410 in a desired rotational position relative to the actuator 414 and a desired translatable position relative to the openings 408.

The proximal portion 404 of the instrument 402 is further shown to include an angel wing 460, with the angel wing 460 releasably couplable with the guide 410. Accordingly, any translation and/or rotation of the guide 410 is also applied to the angel wing 460. The angel wing 460, as shown, includes a substantially elongated (e.g., rectangular) geometry, with a coupling portion 462 compatible with the guide 410 positioned at a first end thereof. The coupling portion 462 may include a threaded actuator or other instrument configured to facilitate coupling with complementary components of the guide 410 disposed in an extension extending upward from a portion of the guide 410. The angel wing 460 is further shown to include at least one indicator 464, shown in FIGS. 23-26, 30, and 36-37 and as a pair of linear radiopaque rods positioned parallel to one another, along a length thereof between the first end and a terminal second end of the angel wing 460. In the coupled configuration as seen in at least FIGS. 26-30, the axes/trajectories of each of the indicators 464 is parallel to a corresponding axis/trajectory of each of the openings 412, with the axes/trajectories of the openings 412 positioned directly below that of the indicators 466. Accordingly, the trajectory of the openings 412 can be viewed over the first metatarsal 202, the medial cuneiform 204, the navicular 206, etc. under fluoroscopy based on the radiopaque property of the indicators 464. The angel wing 460 is further shown to include indicators 466 positioned between the indicators 464 and the terminal end of the angel wing 460. In the exemplary embodiment of FIGS. 23-37, the indicators 466 are shown as four short, vertically-positioned radiopaque rods and a pair of horizontally-positioned short, radiopaque rods positioned distal to the four aforementioned rods. The four vertically positioned rods, when viewed on fluoroscopy, will appear as four equidistantly-spaced dots when viewed from a perfectly superior angle, which allows a physician to ensure proper angulation of the instrument 402 under fluoroscopy. In some aspects, the indicators 466 may be positioned centrally along the length of the angel wing 460 (e.g., in a central third thereof), or may be positioned adjacent a terminal end thereof (e.g., opposite the angel wing 460 from the coupling portion 462). The two horizontally positioned rods, when viewed under fluoroscopy, will appear as two lines perpendicular to the indicators 464 and, similar to the four vertically positioned rods, are indicative of any angulation in the positioning of the instrument 402 under fluoroscopy. Accordingly, a physician may manipulate the guide 410 and/or other components of the instrument 402 so as to adjust the trajectories of the openings 412 based on the indications of the trajectories and the positioning of the instrument 402 based on the indicators 464, 466 of the angel wing 460.

In implementing the instrument 402, a physician may position the guide 410 in a desired rotational and translational position (based on visual feedback from the indicators 464, 466), and manipulate the actuator 414 so as to retain the guide 410 in such a desired position. The physician may then implement one or more instruments, for example sleeves/guides for drills, k-wires, or other instruments, to place a k-wire through one of the openings 412 and into both a first and second segment of the first metatarsal 202 (where the first metatarsal 202 as already been osteotomized at a point along the length thereof, as shown in FIGS. 31-37). Once the k-wire has been placed, the physician may then place a cannulated drill bit over the k-wire and drill along the trajectory of the k-wire so as to guide the path of the dill bit and prevent skiving. After a pilot hole has been created by the drill bit, the physician may insert an implant 302, for example from an implant system 300 such as that shown in FIGS. 16-30, into the pilot hole thus providing fixation of the proximal and distal segments of the first metatarsal 202. In some aspects, the k-wire may be removed prior to the placement of the implant 302. In some aspects, the lateral-most opening of the openings 412 may be drilled and the implant 302 placed therethrough prior to the lateral-most opening 412 being drilled and the implant 302 placed therethrough. In some aspects, a physician may adjust the trajectory of the openings 412 by manipulating the guide 410 and actuator 414 after the first implant 302 is placed but prior to the second implant 302 being placed. For example, the first implant 302 may be placed and, in the instance the physician desires converging or diverging trajectories for the two implants 302, the guide 410 may be rotated and/or translated such that the trajectory of the second opening 412 (e.g., the medial-most opening 412) is oblique relative to an axis of the already-placed implant 302. The physician may then repeat the steps of placing a k-wire through the opening 412 (now the medial-most opening 412), guiding a drill bit over the k-wire, and finally placing an implant 302 within the pilot hole created by the drill bit such that an axis of the implant 302, once placed, is oblique relative to that of the first-placed implant 302. It should be understood that, in some aspects, the implants 302 may be placed with axes thereof parallel to one another (e.g., the guide 410 is not adjusted between placement of the first and second implants 302). Further, it should be understood that the aforementioned steps may be performed in alternate orders. For example, the medial-most opening 412 and implant 302 may be drilled and implanted, respectively, prior to the lateral-most opening 412 and implant 302. Further, both openings 412 may be drilled prior to the implantation of any implants 302.

The distal portion 430 of the instrument 402 is shown to include a body 432 having a substantially L-shaped geometry, where each leg of the L-shape includes a substantial u-shape (e.g., rotated 90-degrees) similar to that of the body 406. The proximal-most leg of the L-shape is shown to be slidably coupled with the body 432 via a pair of rails 420 extending parallel to one another with at least one of the proximal and distal portions 404, 430 slidably coupled with the other. The body 432 is further shown to receive at least a portion of an actuator 418 positioned adjacent to the body 432 and includes a threaded shaft extending from a knob portion. The threaded shaft is shown to extend through a portion of the body 406 and be at least partially received by a portion of the body 432. Accordingly, manipulation of the actuator 418 may translate either the proximal portion 404 or the distal portion 430 toward the other (along the rails 420) as the threaded rod portion of the actuator 418 advances within or withdraws from the threading of the respective opening of the body 406.

The body 432 is further shown to include a pair of openings 434 disposed in the legs of the L-shape of the body 432, with the openings 434 being the open portion of the u-shapes of the legs of the body 432. The body 432 is shown to include a bore positioned distal relative to the proximal opening 434 which includes an actuator 436 extending therethrough. The actuator 436 is shown to include a knob portion on a medial-most side of the body 432 and a threaded portion extending through the bore to the lateral side of the body 432. The threaded portion of the actuator 436 may be translated in the medial-lateral direction by rotating the knob portion of the actuator 436. The actuator 436 is shown to be coupled (but may be integral in some aspects) with an engagement member 438 at a distal end of the threaded portion of the actuator 436. The engagement member 438 includes an opening configured to receive at least a portion of the threaded portion of the actuator 436 therein. Accordingly, translation of the threaded portion of the actuator 436 as a result of manipulation of the knob portion of the actuator 436 similarly translates the engagement member 438. The body 432 is also shown to include a button 439 configured to facilitate adjustment of the engagement member 438 relative to the body 432. For example, a physician may engage the button 439 and, while keeping the button 439 pressed/engaged, reposition at least a portion of the engagement member 438 relative to the body 432 so as to accommodate the anatomy of the first metatarsal 202 of a patient.

The engagement member 438 is shown to include a protrusion extending therefrom in at least one of the proximal and/or lateral directions, shown in FIGS. 32 and 34-35 as a hooked member. The hooked member of the engagement member 438 may be positioned adjacent an osteotomy site of the first metatarsal 202, with the distal-most portion of the hooked member positioned within the intramedullary canal of the proximal segment of the first metatarsal 202 such that at least a portion of the hooked member abuts a wall of the intramedullary canal. Accordingly, the actuator 436 may then be manipulated so as to reposition the proximal segment of the first metatarsal 202 relative to the distal segment of the first metatarsal 202.

The distal portion 430 is further shown to include an adjustment mechanism 442 which is releasably and pivotably coupled with the body 432 via a coupling 440. The coupling 440 is positioned at the terminal end of the opening 434 of the distal-most leg of the L-shape of the body 432 such that the adjustment mechanism 442 is rotatable (e.g., pivotable) about the coupling 440. The adjustment mechanism 442 is also releasably and translatably coupled with the body 432 via an actuator 450 which includes a knob portion and a threaded portion, with at least a portion of the threaded portion received through a threaded opening of the body 432 (e.g., positioned on a portion of the distal-most leg of the L-shape). Accordingly, manipulation of the knob portion of the actuator 450 drives translation of the adjustment mechanism 442 closer/further in the proximal-distal direction relative to the body 432.

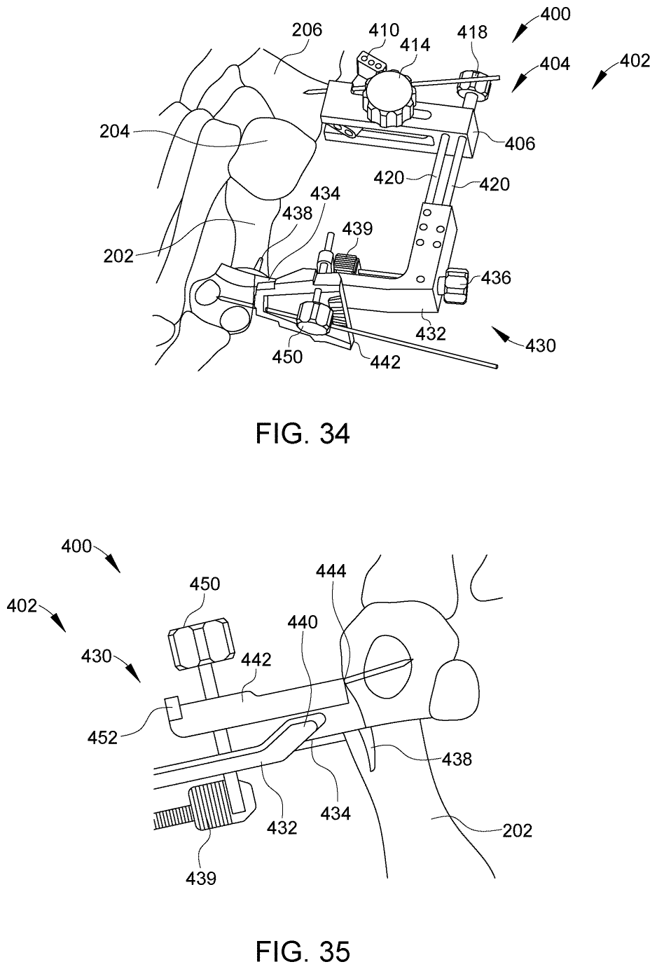

The adjustment mechanism 442 is shown to include an opening 444 at a first end, shown in FIG. 34 as a lateral-most end, and an opening 448 opposite the adjustment mechanism 442 at a second end (shown in FIG. 34 at a medial-most end). The adjustment mechanism 442 further includes one or more slots 446 having an elongated geometry and positioned between the openings 444 and 448. Accordingly, a k-wire may be coupled with the first metatarsal 202 (e.g., a distal segment thereof) and extend through the opening 444, into the adjustment mechanism 442, through one of the slots 446, and out the adjustment mechanism 442 via the opening 448. Further the k-wire may be manipulated by a physician from one of the lower slots 446 to a higher slot 446 so as to correct a rotational deformity of the first metatarsal 202 (and/or a segment thereof). In some aspects, the slots 446 may be labeled, for example with specific angular measurements, to quantify the correction achieved with manipulation of the k-wire from one of the slots 446 to the next. The adjustment mechanism 442 further includes a locking mechanism 452 positioned adjacent the opening 448 configured to retain the k-wire in a desired slot 446 after placement, as the slots 446 are open on one side so as to permit placement therein of the k-wire. The locking mechanism 452 is shown to be pivotably coupled with the adjustment mechanism 442 adjacent the bottom-most portion of the opening 448, and may be rotated (e.g., pivoted about the coupling point) to releasably couple with a point adjacent the upper-most portion of the opening 448 as the k-wire is in the desired slot 446.

In some aspects, a physician may manipulate the distal segment of the first metatarsal 202 via the adjustment mechanism 442 (and a k-wire) and the proximal segment of the first metatarsal 202 via the engagement member 438 to achieve a desired position of the segments. The physician may then manipulate the actuator 436 and/or the locking mechanism 452 to retain the aforementioned segments in the desired position prior to manipulating any components of the proximal portion 404 of the instrument 402 as described previously herein.

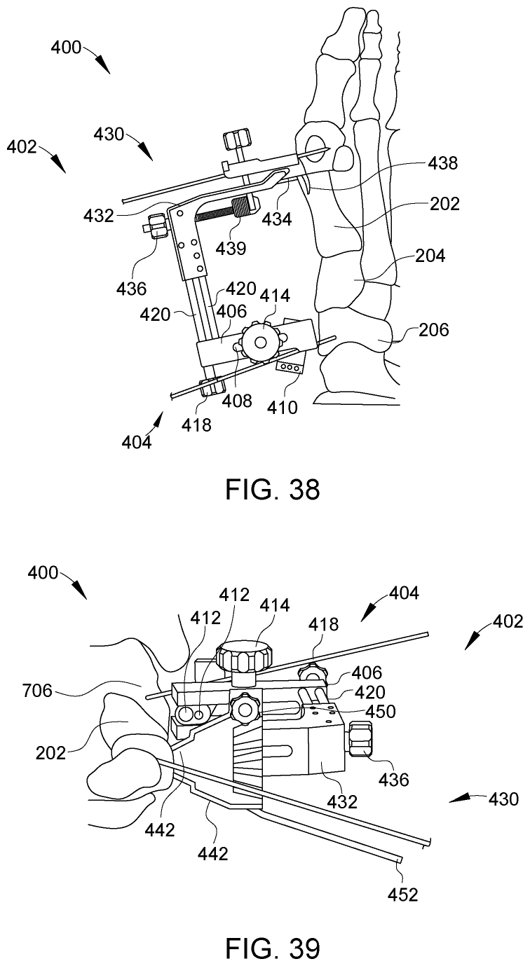

Referring now to FIGS. 38-46, an exemplary surgical method for implementing the system 400 (or other similar systems, for example the system 100) is shown, according to an exemplary embodiment. While the steps shown in FIGS. 38-46 reference the system 400 and components thereof, it should be understood that the steps of the method shown in FIGS. 38-46, individually or collectively as the surgical method, may be applied to surgical systems other than the systems 100, 400 shown and described herein. Further, it should be understood that the surgical method shown in FIGS. 38-46 may be performed in an alternate order, or may be performed with one or more steps repeated and/or omitted.

The steps of the surgical method shown and described with respect to the FIGS. 38-46 are to be performed after the foot of the patient has been prepared for surgery and one or more steps has already been performed. For example, a physician may have created an incision along the medial portion of the midfoot of the patient to allow access to at least the first metatarsal 202 of the patient to perform an osteotomy of the first metatarsal 202 (and thus creating distal and proximal portions of the first metatarsal 202).

Referring now to FIG. 38, the step of coupling the instrument 402 with the anatomy of a patient is shown, according to an exemplary embodiment. The step of FIG. 38, as shown, may include engaging the engagement member 438 and the tip/projection extending therefrom with at least a portion of the first metatarsal 202. Further, the step of FIG. 38 may include positioning the engagement member 438 such that at least a portion of the engagement member 438 is disposed within the intramedullary canal of the proximal portion of the first metatarsal 202. In some aspects, the physician may implement fluoroscopy to guide and/or confirm the aforementioned positioning of the engagement member 438. The physician may also manipulate the actuator 436 to translate the engagement member 438 in the medial-lateral direction as needed relative to the anatomy. Further, in positioning the engagement member 438 as mentioned, the physician may also manipulate the instrument 402 (and the distal potion 430 thereof) such that the adjustment mechanism 442 and the opening 444 is positioned on a medial portion thereof are disposed adjacent the medial surface of the distal fragment of the first metatarsal 202. As shown in FIG. 38, the physician may also position the medial side of the body 406 adjacent to the navicular 206 of the patient. Further, the physician may also releasably couple the body 406 (and thus, the instrument 402) with the navicular 206 using a k-wire as shown in at least FIG. 38.

Further, in performing the step of FIG. 38, the physician may position the instrument 402 such that the pair of rails 420 are substantially parallel to the long axis of the first metatarsal 202, as shown in FIG. 33. Further, while using fluoroscopy to guide this positioning, the physician may manipulate the instrument 402 such that the engagement member 438 can be viewed through one or more openings in the instrument 402. Once the physician has achieved the desired positioning of the instrument,

Referring now to FIG. 39, the physician may manipulate the locking mechanism 452 from a closed position to an open position. Further, the physician may then releasably couple the distal portion 430 of the instrument 402 with the proximal portion segment of the first metatarsal 202 by placing a k-wire through the opening 448 of the adjustment mechanism 442, between two of the slots 446 thereof, and out the opening 444 with the k-wire ultimately being inserted in the medial portion of the distal segment of the first metatarsal 202. The physician may then manipulate the locking mechanism 452 from the open position back to the closed position so as to retain the k-wire in the desired slot 446 of the adjustment mechanism 442.

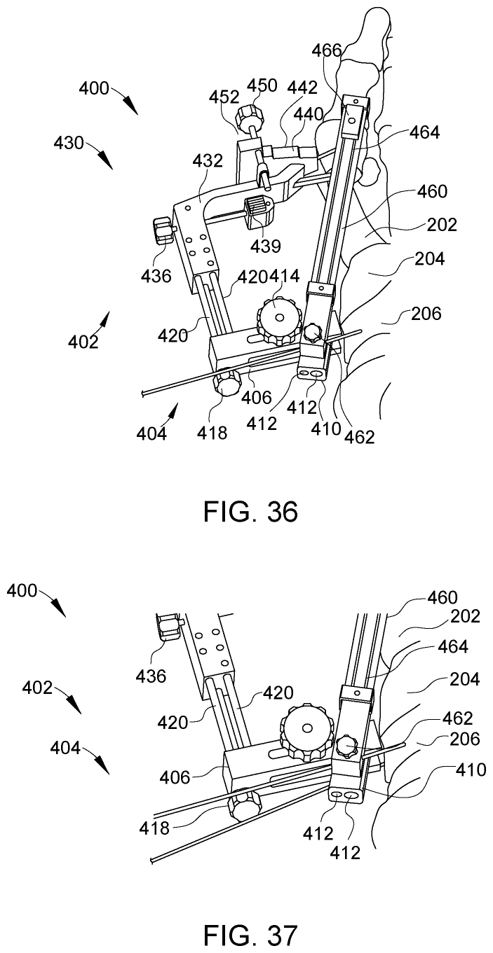

After performing the step of FIG. 39, the physician may releasaby couple the angel wing 460 with the body 406 of the instrument 402 such that the angel wing 460 is positioned superior relative to the foot of the patient (and the angel wing 460 extends over at least a portion of the first metatarsal 202, as shown in FIGS. 36-37). The physician may then manipulate the instrument 402 and/or the angel wing 460 under fluoroscopy so as to achieve a desired position of the instrument 402 and the angel wing 460 based on the positioning of the indicators 464, 466 which are shown to be radiopaque. Once the physician has achieved a desired position of the instrument 402 and the angel wing 460, the physician may releasably couple the body 406 of the instrument 402 with the navicular 206 via a second k-wire (in addition to the first k-wire already coupling the instrument 402 with the navicular 206).

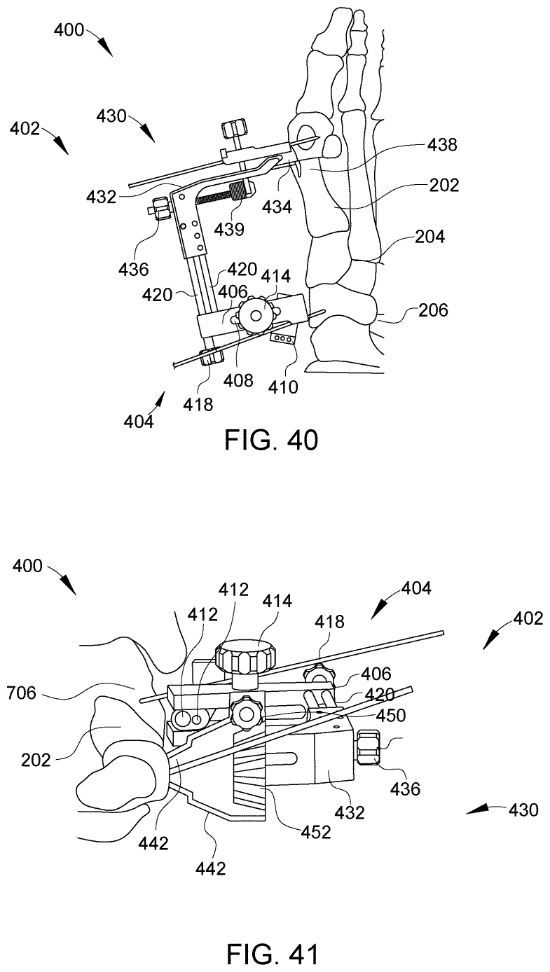

Referring now to FIG. 40, the step of correcting a deformity is shown, according to an exemplary embodiment. In the step shown in FIG. 40, the physician may manipulate the actuator 436 so as to translate the distal component of the first metatarsal 202 in the lateral direction (in the medial-lateral plane). By rotating the actuator 436, the instrument 402 maintains the proximal portion of the first metatarsal 202 in a static position while shifting the distal portion of the first metatarsal 202 laterally. The physician may perform this translation under fluoroscopy and/or may use fluoroscopy to determine when the distal portion of the first metatarsal 202 has reached a desired position.

Referring now to FIG. 41, the step of correcting a deformity is shown, according to an exemplary embodiment. In performing the step seen in FIG. 41, the physician may manipulate the locking mechanism 452 of the adjustment mechanism 442 from a closed position to an open position. The physician may then grasp the k-wire positioned within and extending from the adjustment mechanism 442, and manipulate the k-wire from an initial position in a first slot of the slots 446 to a second position in a second slot of the slots 446. In some aspects, the second slot of the slots 446 may be higher than the first slot of the slots 446. In some aspects, the physician may position the k-wire in a third slot after positioning the k-wire in the second slot, for example if the physician determines (e.g., using fluoroscopy) that the rotation of the distal portion of the first metatarsal 202 achieved by positioning the k-wire in the second slot does not position the distal portion of the first metatarsal 202 in a desired position. Once the physician has positioned the k-wire in a slot of the slots 446 that corresponds with a desired corrected position (e.g., rotationally corrected) of the distal portion of the first metatarsal 202, the physician may manipulate the locking mechanism 452 from the open position to the closed position so as to retain the k-wire in the desired slot and maintain correction of the distal portion of the first metatarsal 202.

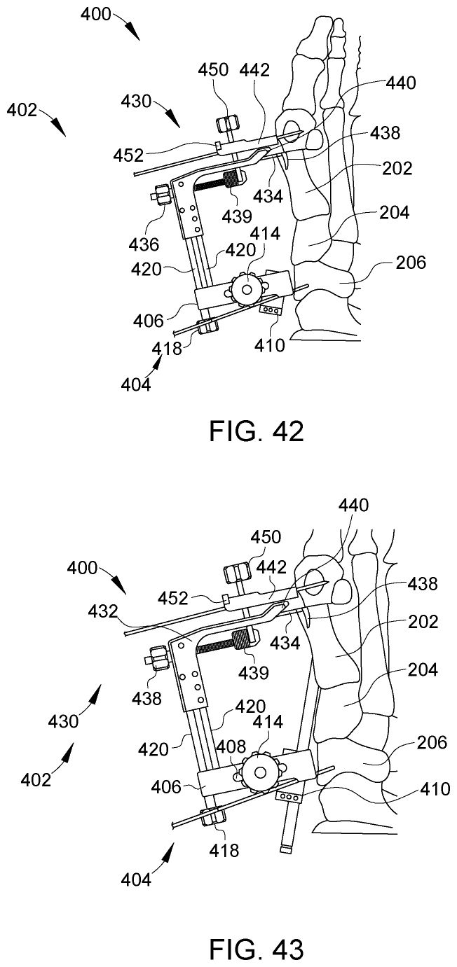

Referring now to FIG. 42, a step of deformity correction is shown, according to an exemplary embodiment. In performing the step shown in FIG. 42, the physician may manipulate the actuator 450 so as to correct a deformity of the distal metatarsal articular angle (DMAA). In manipulating the actuator 450, the position of the distal portion of the first metatarsal 202 may be adjusted relative to the position of the proximal portion of the first metatarsal 202. For example, the distal portion of the first metatarsal 202 may be positioned, by rotation of the actuator 450, such that maximum surface area of the proximal surface created by the osteotomy is in a plane parallel to and/or in contact with a maximum surface area of the distal surface of the proximal portion of the first metatarsal (also created by the osteotomy). In some aspects, the physician may also address any gapping between the adjacent surfaces created by the osteotomy (e.g., the distal surface of the proximal portion and the proximal surface of the distal portion of the first metatarsal 202) in the step of FIG. 42. In doing so, the physician may manipulate the actuator 450 so as to bias the distal and proximal portions of the first metatarsal 202 closer to one another such that the aforementioned adjacent surfaces abut one another.

Referring now to FIG. 43, the step of targeting for an implant 302 is shown, according to an exemplary embodiment. In performing the step seen in FIG. 43, a physician may insert a tube (e.g., a tissue protector) into and through one of the openings 412. As shown, the tube is inserted through the lateral-most of the openings 412, but in some aspects the physician may elect to insert the tube in the medial-most of the openings 412. As shown in FIG. 43, the distal portion (e.g., tip) of the tissue protector is shown to contact the proximal portion of the first metatarsal 202 near the medial cuneiform 204. Further, the tube is shown to be along an axis and trajectory parallel to that of one of the indicators 464 of the angel wing 460. A drill guide may then be placed within the tube, with the drill guide having a cannulation. Once the drill guide is placed, the physician may insert a k-wire within the drill guide and advances the k-wire into the proximal portion and then the distal portion of the first metatarsal 202.

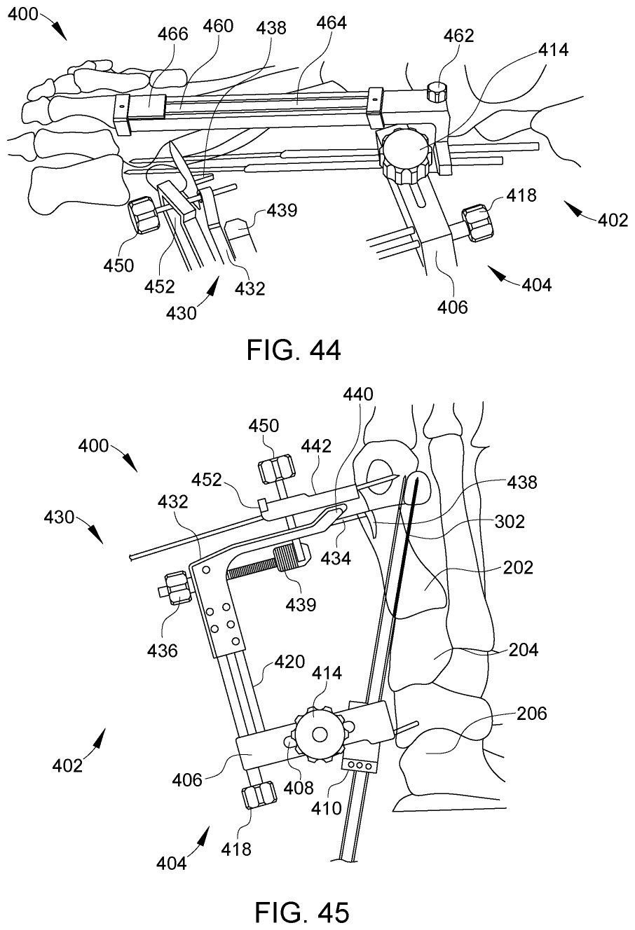

Referring now to FIG. 44, a step of targeting for an implant 302 is shown, according to an exemplary embodiment. In performing the step shown in FIG. 44, the physician may position a second drill guide through the lateral-most (e.g., left) of the openings 412. This opening is smaller in diameter than the medial-most opening, and thus the opening doesn't require the tissue protector therein. Similar to the step of FIG. 43, the physician may then advance a k-wire through the proximal and then distal portions of the first metatarsal 202, with said k-wire positioned medially relative to the k-wire of the step of FIG. 43.

Referring now to FIG. 45, the step of placing an implant 302 is shown, according to an exemplary embodiment. In performing the step seen in FIG. 45, the physician may remove the tube and drill guide from the lateral-most opening 412, while leaving the k-wire positioned therein. The physician may then position a cannulated implant, for example the implant 302, such that the k-wire is positioned at least partially within said cannulation, and advance the implant 302 along the trajectory of the k-wire. Accordingly, the implant 302 is advanced through the proximal portion of the first metatarsal 202 and into the distal portion of the first metatarsal 202. The k-wire is then removed after the implant 302 has been placed.

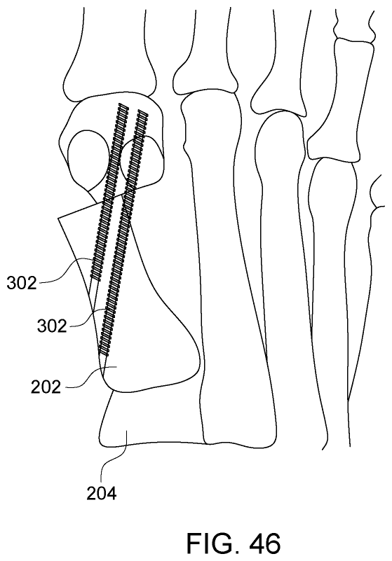

Referring now to FIG. 46, the step of placing an implant is shown, according to an exemplary embodiment. In performing the step of FIG. 46, the physician may remove the drill guide from the medial-most opening 412, while leaving the k-wire positioned therein. The physician may then position a cannulated implant, for example the implant 302, such that the k-wire is positioned at least partially within the cannulation, and advance the implant 302 along the trajectory of the k-wire. Accordingly, the implant 302 is advanced through the proximal portion of the first metatarsal 202 and into the distal portion of the first metatarsal 202. The k-wire is then removed after the implant 302 has been placed. Once the implant 302 has been placed, the pair of implants 302 are positioned along parallel trajectories. However, in some aspects the physician may wish to place the implants 302 along converging or diverging trajectories. In order to do so, the physician may complete the steps of FIGS. 43-46 for one of the implants 302, then reposition the instrument 402 (e.g., using the angelwing 460 once again) to target a desired trajectory that is converging or diverging relative to that of the already-placed implant 302. The physician may then perform the steps of FIGS. 43-46 for the remaining implant 302.

The terminology used herein for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. An instrument, comprising:
a proximal portion, comprising:
a guide comprising at least one opening, the at least one opening comprising at least one trajectory; and
a distal portion, comprising:
an engagement mechanism configured to engage with a first segment of a first metatarsal of a patient; and
an adjustment mechanism configured to engage with a second segment of the first metatarsal of the patient.

2. The instrument of claim 1, wherein the proximal portion of the instrument comprises:
a pair of legs extending substantially parallel to one another and comprising a top leg and a bottom leg, wherein the top leg comprises an elongated opening positioned along a length of the top leg and extending from a top surface of the top leg through to a bottom surface of the top leg.

3. The instrument of claim 2, wherein the guide comprises at least one protrusion extending outwardly therefrom.

4. The instrument of claim 3, wherein the at least one protrusion comprises a lateral dimension lesser than that of the elongated opening of the top leg of the proximal portion of the instrument.

5. The instrument of claim 4, wherein the guide comprises a cannulation along a longitudinal axis of the at least one protrusion and extending at least partially through the at least one protrusion.

6. The instrument of claim 5, further comprising an actuator configured to releasably couple with the cannulation of the at least one protrusion of the guide.

7. The instrument of claim 6, wherein at least a portion of the at least one protrusion of the guide is positioned within the elongated opening of the top leg so as to enable translation of the guide along the length of the elongated opening.

8. The instrument of claim 7, wherein the guide is pivotable about the longitudinal axis of the at least one protrusion.

9. The instrument of claim 8, wherein manipulation of the actuator in a first direction permits translation of the guide along the length of the elongated opening, and manipulation of the actuator in a second direction secures the guide at a position along the length of the elongated opening.

10. The instrument of claim 9, wherein manipulation of the actuator in a first direction permits pivoting of the guide about the longitudinal axis of the at least one protrusion, and manipulation of the actuator in a second direction restrains pivoting of the guide at a rotational position about the longitudinal axis of the at least one protrusion.

11. The instrument of claim 10, wherein the engagement mechanism comprises an L-shaped component having a first segment and a second segment separated by a substantially 90-degree angle, wherein the second segment comprises a textured surface along at least a portion of a length between the substantially 90-degree angle and a terminal end opposite the second segment from the substantially 90-degree angle.

12. The instrument of claim 11, wherein at least a portion of the second segment of the engagement mechanism is configured to engage with an intramedullary canal of the first segment of the first metatarsal.

13. The instrument of claim 12, wherein the distal and proximal portions of the instrument are separated by a body, wherein the body comprises a pair of rails along which the distal portion may be translated relative to the proximal portion of the instrument.

14. The instrument of claim 13, wherein the adjustment mechanism is pivotable relative to a distal-most portion of the distal portion of the instrument.

15. The instrument of claim 14, wherein the adjustment mechanism further comprises:
a first opening at a first end;
a plurality of slots opposite the adjustment mechanism from the first end;
a second opening adjacent the plurality of slots; and
a locking gate positioned adjacent the second opening.

16. The instrument of claim 15, further comprising a fixation wire, wherein the fixation wire is configured to extend through the second opening, through one of the plurality of slots, through the first opening, and into the second segment of the first metatarsal.

17. The instrument of claim 16, wherein the locking gate is moveable between a locked and an unlocked position, wherein in the unlocked position the fixation wire may be manipulated from a first slot of the plurality of slots to a second slot of the plurality of slots while extending through the first and second openings of the adjustment mechanism.

18. An instrument, comprising:
a proximal portion, comprising:
a guide translatable about an elongated opening and pivotable about a longitudinal axis thereof, the guide comprising:
at least one opening, the at least one opening comprising at least one trajectory; and
a distal portion, comprising:
an engagement mechanism configured to engage with a first segment of a first metatarsal of a patient, wherein the engagement mechanism comprises:
a first segment; and
a second segment separated from the first segment by a substantially 90-degree angle, wherein the second segment comprises a texture along at least a portion of a length thereof; and
an adjustment mechanism configured to engage with a second segment of the first metatarsal of the patient, wherein the adjustment mechanism comprises:
a first opening;
a plurality of slots;
a second opening; and
a locking gate.

19. The instrument of claim 18, wherein the proximal and distal portions are separated by a body comprising at least one rail, wherein the distal portion is translatable relative to the proximal portion.

20. A surgical method comprising:
coupling an instrument to the foot of a patient;
positioning the instrument such that a pair of rails are substantially parallel to a long axis of a first metatarsal;
manipulating a locking mechanism of the instrument from a closed position to an open position;
coupling a distal portion of the instrument with a proximal portion segment of the first metatarsal;
correcting a deformity of the first metatarsal;
targeting for the implantation of at least one implant; and
implanting the at least one implant into the first metatarsal.
